(19) United States
(12) Patent Application Publication
(10) Pub. No.: US 2019/0127795 A1
(43) Pub. Date: May 2, 2019

(54) METHOD FOR PROGNOSING AND REDUCING CARDIOVASCULAR DISEASE IN PATIENTS WITH KIDNEY DISEASES

(71) Applicants: TIFFANY WU, NEW YORK, NY (US); Chi-Hua Foundation, New Taipei City (TW)

(72) Inventor: WAN-LIN WU, New Taipei City (TW)

(73) Assignee: TIFFANY WU, NEW YORK, NY (US)

(21) Appl. No.: 15/926,760

(22) Filed: Mar. 20, 2018

Related U.S. Application Data
(60) Provisional application No. 62/485,369, filed on Apr. 13, 2017.

(51) Int. Cl. C12Q 1/6883; C12N 15/113

(52) U.S. Cl. CPC C12Q 1/6883; C12N 15/113; C12N 2310/341; C12Q 2600/118; C12Q 2600/158

(57) ABSTRACT

The present invention discloses a method for prognosing and reducing cardiovascular diseases in patients with kidney diseases, comprising obtaining a bio-sample of a healthy person and a bio-sample of a patient with kidney disease, detecting the expression of specific lncRNA biomarkers individually, such as one or more combinations of DKFZP434I0714, KCNJ2AS1, LOC256880, LOC644656, FAM86FP, FAM66D, LOC100289511, and HTR7P1, comparing the expressions between the samples of the healthy persons and the samples of the patients with kidney diseases to obtain a ratio, and prognosing whether the patients with kidney diseases belong to a high-risk group to have cardiovascular diseases based on the ratio. Furthermore, the method reduces the possibility of patients with kidney diseases to have cardiovascular diseases through the inhibition technology.

### BACKGROUND OF THE INVENTION

### Field of the Invention

The present invention relates to a method for prognosing and reducing cardiovascular disease in patients with kidney diseases, especially the biomarker provided by the present disclosure may prognose the risk of having cardiovascular disease in patients with kidney diseases, and reduce the chance to have cardiovascular disease in patients with kidney diseases through inhibiting the biomarker.

### Description of The Related Art

In the recent ten years, the population of the patients with chronic kidney disease has been increasing rapidly worldwide, and it has gradually become a serious medical and social issue. In Taiwan, nearly seventy thousand people require dialysis treatment to maintain kidney function every year. Per statistic, the most common reason of deaths in the patients having hemodialysis is cardiovascular disease. Fifty percentage of the patients with kidney diseases at terminal stage have cardiovascular diseases, and 80% of those patients with kidney diseases at terminal stage under the treatment of hemodialysis have cardiovascular diseases.

Patients with chronic kidney diseases have the symptoms of raising the concentration of inflammation substance in their serum, and these inflammation reactions cause: 1. Increased death rate, wherein the risk of a death caused by cardiovascular disease is higher than normal people; 2. Higher risk of having cardiovascular diseases, including peripheral artery occlusive diseases, coronary artery disease and cerebrovascular arteriosclerosis; 3. deterioration of kidney function; 4. Increased complications of chronic kidney diseases, such as, anemia, insulin resistance and renal osteodystrophy; and 5. Poor appetite, malnutrition and weight loss.

Inflammation may induce the generation of cytokines (TNF-α, IL-1β, etc.), cell adhesion molecules (VCAM-1, ICAM-1, E-selectin, etc.), chemokine (MCP-1), and inflammation-related proteins, wherein the cell adhesion molecules expressed by endothelial cells may facilitate the adhesion of monocyte cells and endothelial cells. Monocyte cells may further penetrate the endothelial cells into the inner membrane of vessel, and become macrophage. The macrophage unlimitedly intakes a huge amount of oxidized low-density lipoprotein cholesterol to form foam cells. The process is being considered as a primary and critical step in the formation of atherosclerosis. Therefore, the cell adhesion molecules may be used as an indicator to prognose cardiovascular diseases in the current research.

50% of the patients having hemodialysis die because of cardiovascular diseases, including sudden cardiac arrest, acute myocardial infraction, arrhythmia and other forms of cardiovascular events. Patients with chronic kidney diseases and uremic patients undergoing hemodialysis commonly face the issues comprising hypertension, ectopic calcification, bone disease, uremic toxins, chronic inflammation, and high blood sugar, etc., and further with instability of dyslipidemia or vascular plaque, thereby increasing the risk of cardiovascular diseases. Therefore, the researchers in this field all devote to find reliable biomarkers to facilitate early diagnosis and to predict the prognosis of cardiovascular diseases in patients with chronic kidney diseases and uremia.

Long Non-Coding RNAs (lncRNAs) are a class of RNA transcripts longer than 200 bp that are not translated into proteins. In recent years, studies have suggested that lncRNAs are involved in the regulation of cellular function, and lncRNAs function critically in regulating gene expression, maintaining genome integrity, compensating gene dosage, genome imprinting, mRNA processing, and cell differentiation and development. Aberrantly expressed lncRNAs contribute to the development of many diseases including cancers, immune diseases and neurological disorders, would occur.

Endothelial nitric oxide synthase (eNOS) may generate nitric oxide (NO), which is important for normal endothelial and vascular function. The amount of NO in the vasculature is altered with atherosclerosis and many cardiovascular diseases. Therefore, the abnormality of eNOS gene expression may affect the risk of atherosclerosis in an individual. Some people use eNOS as a diagnostic marker for atherosclerosis.

Because eNOS functions by generating NO in blood vessel, eNOS is one of the most attractive therapeutic target for cardiovascular diseases.

Krüppel-like transcription factors 2 (KLF2) belongs to transcription factors of Krüppel family. KLF2 may improve the movement of perivascular cells and smooth muscle cells in the late stage of vessel development to form vessel wall structure, further to concrete neovascularization. KLF2 are involved in the maintenance of normal vascular functions, such as anti-inflammation, anticoagulation, vasodilation, vascularization and regulation of endothelial cell secretion, wherein in the anticoagulation and vasodilation, KLF2 increases the expression of eNOS to maintain normal physiological functions. In various vascular diseases, such as atherosclerosis, diabetes and transient ischemic attack, the expression of KLF2 deceases. Therefore, increasing the expression of KLF2 may be considered as a method to treat vascular diseases.

### SUMMARY OF THE INVENTION

The purpose of the present disclosure provides a method for prognosing and reducing cardiovascular diseases in patients with kidney diseases. According to each and every research result, it is confirmed that the screened lncRNA of the present disclosure may prognose patients with kidney diseases belonging to a high-risk group to have cardiovascular diseases.

The achieve the aforementioned purpose, the technique is analyzing the expression of the specific target, lncRNA, in the blood sample of the patients with kidney disease, such as one or more combinations selected from DKFZP43410714, KCNJ2AS1, LOC256880, LOC644656, FAM86FP, FAM66D, LOC100289511 and HTR7P1. If the expression of the target, lncRNA, in the patients with kidney diseases is higher than the average, the patient belongs to the high-risk group of having cardiovascular diseases in the future.

The other purpose of the present disclosure provides a method for reducing cardiovascular diseases in patients with kidney diseases.

To accomplish the aforementioned purpose, the technical feature is utilizing the technology of antisense DNA oligos or similar agents to inhibit the expression of target lncRNAs in patient with kidney diseases, in order to decrease possibility of patient with kidney diseases to have cardiovascular diseases .

### BRIFE DESCRIPTION OF THE DRAWINGS

The patent or application file contains at least one drawing executed in color. Copies of this patent or patent application publication with color drawing(s) will be provided by the Office upon request and payment of the necessary fee.

The detailed description of the drawings particularly refers to the accompanying figures in which:

FIG. 1 shows the comparison of lncRNA expression in plasma sample between patients with kidney diseases at terminal stage who have cardiovascular diseases and patients with kidney diseases at terminal stage who have no cardiovascular diseases.

FIG. 2 shows the results of the expression of target lncRNA in Embodiment 2 as in bitmap.

FIG. 3 shows Kaplan-Meier curve of the expression of biomarker DKFZP43410714 in patients with kidney disease and survival times in Embodiment 3.

FIG. 4 shows the results of the inhibition to DKFZP43410714 in Embodiment 4.

### DETAILED DESCRIPTION OF THE INVENTION

For a better knowledge and understanding of the present disclosure as a courtesy for the examiner, the technical features and process of the present disclosure have been illustrated by the embodiments and drawings below.

### Embodiment 1

The present disclosure provides a method for prognosing and reducing cardiovascular diseases in patients with kidney diseases, comprising steps of:

The plasma from blood sample in test sample of the present embodiment, and these samples have been tracked for five years and collected:

Sample A: Healthy people, 13 counts;

Sample B: Patients with kidney failure, 19 counts;

Sample C: Patients with kidney diseases at terminal stage who have no cardiovascular diseases, 43 counts; and

Sample D: Patients with kidney diseases at terminal stage who have cardiovascular diseases, 36 counts.

Firstly, isolate RNA from plasma sample, and utilize RNA Sequencing technology to analyze the difference between each sample, and analyze potential biomarkers. Furthermore, utilize polymerase chain reaction and analytic software to analyze. The software used in the present embodiment is Multiple Experiment Viewer (MeV).

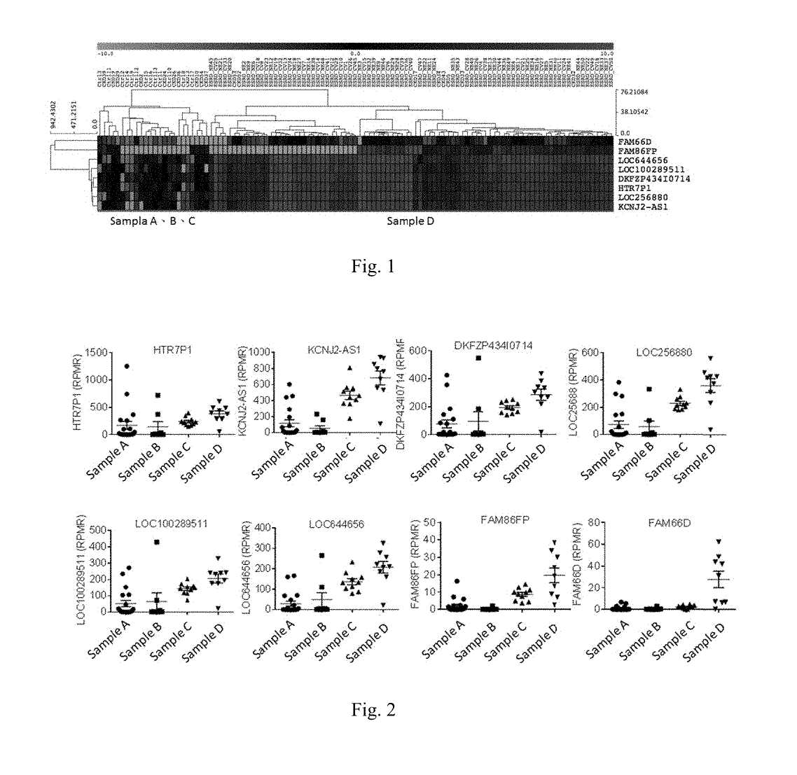

Please refer to FIG. 1, after comparison, there are 8 lncRNA expressions in Sample D, which has significant and stable difference from Samples A, B and C, and these are DKFZP43410714, KCNJ2AS1, LOC256880, LOC644656, FAM86FP, FAM66D, LOC100289511, and HTR7P1.

### Embodiment 2

To confirm that the biomarker lncRNA acquired from Embodiment 1 is positively correlated with patients with kidney diseases having cardiovascular diseases, a further expression analysis is being conducted on eight specific lncRNAs in Embodiment 1.

Please refer to FIG. 2, the expressions of the eight lncRNAs in Sample D are significantly higher than the other three groups. Therefore, when the expressions of the eight lncRNAs in the blood sample of the patients with kidney diseases are significantly higher than healthy people, it is determined that the patients with kidney diseases belong to the high-risk group of having cardiovascular diseases.

Then, analyzing AUC value, sensitivity and specificity of the eight specific lncRNAs, the results is shown in Table 1. The AUC value is roughly above 0.79, and the sensitivity and specificity are higher than 75% except for HTR7P1.

TABLE 1
Analysis of AUC value, sensitivity, and specificity

| | | RPC | | | | | |
|---|---|---|---|---|---|---|---|
| | Average | Analysis | Cutoff | Sensitivity | Specificity | Hazard Ratio | |
| Plasma lncRNAs | RPMR | (AUC) | RPMR | (%) | (%) | (HR, 95% CI) | P value |
| DKFZP434I0714 | 13.6 | 0.93 | 4.0 | 88.9 | 83.3 | 13.62(1.69-109.63) | 0.002 |
| KCNJ2AS1 | 164.4 | 0.88 | 165.6 | 88.9 | 83.3 | 14.71(1.82-118.73) | 0.001 |
| LOC256880 | 226.5 | 0.85 | 226.4 | 77.8 | 75.0 | 4.89(1.01-23.68) | 0.003 |
| LOC644656 | 291.1 | 0.85 | 281.7 | 88.9 | 83.3 | 14.71(1.82-118.70) | 0.001 |
| FAM86FP | 277.3 | 0.86 | 231.6 | 88.9 | 75.0 | 3.98(1.16-13.67) | 0.018 |
| FAM66D | 162 | 0.84 | 159.7 | 88.9 | 83.3 | 14.71(1.82-118.73) | 0.001 |
| LOC100289511 | 541.1 | 0.82 | 523.1 | 88.9 | 75.0 | 11.05(1.37-88.90) | 0.005 |
| HTR7P1 | 13.2 | 0.79 | 9.8 | 66.7 | 66.7 | 2.40(0.60-9.63) | 0.200 |

### Embodiment 3

The present embodiment tracks the correlation between the present biomarker, lncRNA, in the patients with kidney diseases and the survival ratio for a long period of time.

The present embodiment observes the correlation between the expression of lncRNA, DKFZP434I0714, in the patients with kidney diseases (n=98) and its survival ratio of the patients at designated time in fifty months. STATA 14.0 (StataCorp, Tex.) is utilized to perform the survival analysis, and the results is represented using Multivariate Cox regression analysis. The survival ratio of the present embodiment is adverse cardiovascular events or death caused by cardiovascular diseases in patients with kidney diseases.

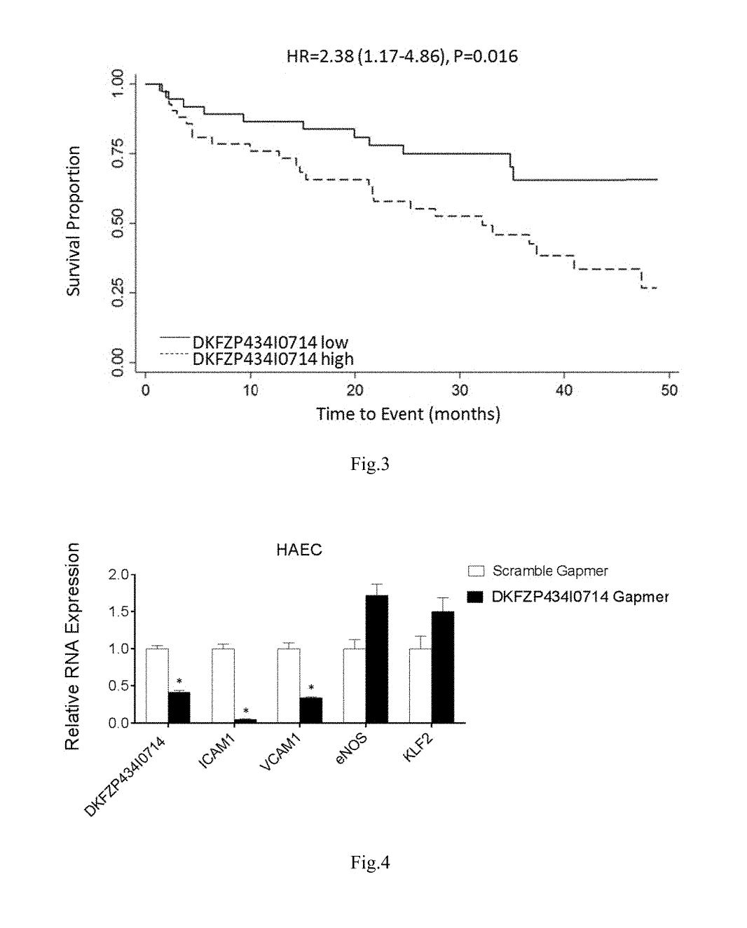

Please refer to FIG. 3, the expression of lncRNA, DKFZP43410714, is closely associated with the survival ratio of the patients with kidney diseases to cardiovascular diseases. During these fifty months, when the expression of lncRNA DKFZP43410714 increases, the survival ratio of the patients with kidney diseases decreases; when the expression of lncRNA, DKFZP43410714, decreases, the survival ratio of the patients with kidney diseases is relatively higher.

To summarize the aforementioned results, the applicants deem: when the expressions of the eight lncRNAs, including DKFZP43410714, KCNJ2AS1, LOC256880, LOC644656, FAM86FP, FAM66D, LOC100289511 and HTR7P1, from patients with kidney diseases increases, the patients with kidney diseases belong to the high-risk group to have cardiovascular diseases. Therefore, the eight lncRNAs may be used as a biomarker to determine whether the patients with kidney diseases belong to the high-risk group to have cardiovascular diseases.

The experimental method to determine expression of eight lncRNAs can be performed with, but not limited to: reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), in situ hybridization (ISH), mass spectrometry (MS), RNA pull-down and the like.

### Embodiment 4

The present invention provides a method for reducing cardiovascular diseases in patients with kidney diseases, and its technique is a known gene inhibition technology, locked nucleic acid (LNA) Gapmer. The present embodiment utilizes LNA Gapmer to inhibit the expression of lncRNA DKFZP434I0714 in human aortic endothelial cells, HAEC, and determine the expressions of known cardiovascular disease factors, ICAM1 and VCAM1, and the expressions of eNOS and KLF2 simultaneously.

Please refer to FIG. 4, after the treatment with DKFZP434I0714-targeting LNA Gapmer, the expressions of lncRNAs, DKFZP434I0714, ICAM1 and VCAM1, have been inhibited and statically meaningful. Furthermore, with the expressions of eNOS and KLF2 significantly increase, it indicates that after the inhibition of the present target, lncRNA, the expression of cardiovascular disease factor has also been inhibited, and the expression of the factor which facilitates vasodilation has increased, the chance of cardiovascular diseases may be decreased.

The aforementioned descriptions are preferable embodiments of the present invention. However, these embodiments are not used as a limitation to the scope of claims of the present invention. The equal application or modification which falls in the scope of the present invention is included in the scope of the present application.

### Description of Symbol

| | |
|---|---|
| Sample A | Healthy people |
| Sample B | Patients with chronic kidney diseases who have not |
| | cardiovascular diseases in five years |
| Sample C | Patients with end-stage renal diseases who have no |
| | cardiovascular diseases in five years |
| Sample D | Patients with end-stage renal diseases who have |
| | cardiovascular diseases in five years |

### Domestic Priority

The present application is the non-provisional application of a provisional application No. 62/485,369

What is claimed is:

1. A method for prognosing cardiovascular diseases in patients with kidney diseases, comprising:
obtaining a sample of a healthy person and a sample of a patient with kidney disease,
detecting expressions of lncRNA biomarkers individually, wherein the biomarker lncRNA is selected from one or more combinations of DKFZP434I0714, KCNJ2AS1, LOC256880, LOC644656, FAM86FP, FAM66D, LOC100289511, and HTR7P1,
comparing the expressions between the sample of the healthy persons and the sample of the patients with kidney diseases to obtain a ratio, and
prognosing whether the patients with kidney diseases belong to a high-risk group to have cardiovascular diseases based on the ratio, wherein the ratio is positively correlated with the high-risk group to have cardiovascular diseases.

2. The method of claim 1, wherein the sample is blood sample.

3. The method of claim 2, wherein the blood sample is plasma.

4. A method for decreasing cardiovascular diseases in patients with kidney diseases, comprising:
utilizing a gene inhibition technology to inhibit expression of a biomarker in human body which causes patients with kidney diseases to have cardiovascular diseases, wherein the biomarker is selected from one or more combinations of DKFZP434I0714, KCNJ2AS1, LOC256880, LOC644656, FAM86FP, FAM66D, LOC100289511 and HTR7P1, and
reducing the risk to have cardiovascular diseases in the patients with kidney diseases.
